(19) United States
(12) Patent Application Publication
(10) Pub. No.: US 2025/0262185 A1
(43) Pub. Date: Aug. 21, 2025

(54) METHODS OF ADMINISTERING NEUROPSYCHIATRIC MEDICATIONS BASED ON RENAL IMPAIRMENT

(71) Applicant: Sumitomo Pharma America, Inc., Marlborough, MA (US)

(72) Inventors: Seth Cabot HOPKINS, Northborough, MA (US); Gerald R. GALLUPPI, Orlando, FL (US)

(73) Assignee: Sumitomo Pharma America, Inc., Marlborough, MA (US)

(21) Appl. No.: 19/053,477

(22) Filed: Feb. 14, 2025

Related U.S. Application Data
(60) Provisional application No. 63/554,269, filed on Feb. 16, 2024.

(51) Int. Cl. A61K 31/381; G16H 20/10

(52) U.S. Cl. CPC A61K 31/381; G16H 20/10

(57) ABSTRACT

Neuropsychiatric medications, particularly ulotaront, and their use based on renal function.

### CROSS REFERENCE TO RELATED APPLICATIONS

This application is a U.S. Non-Provisional application which claims priority to U.S. Provisional Application No. 63/554,269 filed Feb. 16, 2024, the contents of which are hereby incorporated by reference herein in their entirety.

### FIELD

The present disclosure relates to neuropsychiatric medications, particularly ulotaront, and their use based on renal function.

### BACKGROUND

Renal function can play a large role in altering the pharmacokinetics of medications, especially in elimination or clearance and plasma protein binding. Specifically, renal impairment decreases the plasma protein binding secondary to decreased albumin and retention of urea, which competes with medications to bind to the protein. Levy 1977. If a drug is primarily excreted by the kidneys, elimination could be significantly altered in renally impaired patients, particularly those who are severely renally impaired or with end stage renal disease or kidney failure. Absorption, distribution, metabolism and excretion (ADME) studies are typically conducted during drug development to evaluate this potential.

Several reviews have been published discussing the need for dose reduction of antipsychotics when treating renally impaired patients. Gardner 2014, for example, surveyed 43 practicing physicians, and found that 53% would recommend a dose reduction when initiating antipsychotic therapy in renally impaired patients, with a median recommended reduction in dose of 30%. Cohen 2004 reviewed the use of psychotropic medications in the context of impaired renal function, altogether reviewing more than 200 citations, along with product safety and research information from pharmaceutical manufacturers and concluded that most “psychotropic medications are fat soluble, easily pass the blood-brain barrier, are not dialyzable, are metabolized primarily by the liver, and are excreted mainly in bile. Consequently, the majority of these drugs can be safely used with the end-stage renal disease population.”

Ulotaront is a new neuropsychiatric medication having a broad psychiatric profile, which is currently in phase II and III clinical studies for the treatment of schizophrenia, depression, and anxiety. Ulotaront has a mode of action which is different from the current class of antipsychotics in that it does not involve binding at the dopamine D₂ receptor but has a mode of action which relies primarily on its trace amine-associated receptor 1 (TAAR1) agonist activity. Unlike the current class of conventional antipsychotics it does not have a significant affinity for different receptors and does not significantly interact with receptors known to confer side effects of atypical antipsychotics (e.g., D2, α1, M1, H1, 5-HT_{2c}).

Ulotaront has undergone extensive metabolic and pharmacokinetic evaluation in phase I and phase II human clinical studies. Galluppi 2021 evaluated the population pharmacokinetics (PK) of ulotaront in adult subjects using pooled data from seven phase I studies, one phase II acute schizophrenia study, and one 6-month extension study, involving single and multiple (up to 7 days) oral doses (5-150 mg/day) in both healthy adult subjects and adult patients with schizophrenia. Ulotaront was well-absorbed and exhibited dose-proportionality in doses ranging from 10 to 100 mg. Only moderate interindividual variability was observed in concentration-time profiles. No clinically meaningful effects on ulotaront PK parameters were observed based on race, age, sex, formulation (capsule or tablet), or clinical status (healthy volunteer vs. patient with schizophrenia); body weight was the only meaningful covariate.

Xiao 2022 characterized the in vitro absorption, distribution, metabolism and excretion (ADME) properties, preclinical PK, and drug-drug interaction (DDI) potential of ulotaront and its major metabolite, SEP-383103. According to the authors, ulotaront has high solubility, high permeability, and low binding to plasma proteins, and is metabolized via NADPH-dependent and NADPH-independent pathways, with CYP2D6 as the major metabolizing enzyme. With regard to renal elimination, the authors reported that “ulotaront elimination in humans is mostly via hepatic clearance, with renal clearance contributing approximately 15% of total clearance.” There was no evidence of SEP-383103 accumulation in the ADME studies reported by Xiao 2022.

Despite this body of knowledge, a need still exists for a more complete understanding of the metabolism of antipsychotic medications and their metabolites, particularly for ulotaront, to better inform the safe and effective administration of ulotaront, particularly in renally impaired patients.

### SUMMARY

While investigating the ADME of ulotaront in renally impaired patients, the inventors have determined that ulotaront can be administered to some renally impaired patients, and that the dose of ulotaront does not need to be reduced in renally impaired patients to accommodate impaired excretion of ulotaront. This is consistent with the report by Xiao 2022 that renal clearance of ulotaront only accounts for approximately 15% of total clearance. However, below a certain threshold of renal impairment, ulotaront should not be administered at all. It has been unexpectedly discovered that a major metabolite of ulotaront, SEP-383103, is not as readily excreted in renally impaired patients as ulotaront, particularly in patients with severe renal impairment or kidney failure. As a consequence, SEP-383103 accumulates, and exposure to SEP-383103 rises over time when ulotaront is chronically administered and can eventually reach levels exceeding those studied and deemed safe during preclinical toxicology studies.

Accordingly, in one embodiment, the disclosure provides a method of administering ulotaront to a human subject in need thereof with renal impairment, comprising: (a) measuring or evaluating the subject's renal function; and (b) administering ulotaront to the subject if the subject has a creatinine clearance rate greater than or equal to 30 mL/min or an eGFR greater than or equal to 30 mL/min/1.73 m².

In another embodiment, the disclosure provides a method of administering ulotaront to a human subject in need thereof, wherein the subject is treatment naïve or on a preexisting regimen of neuropsychiatric therapy, comprising: (a) measuring or evaluating the subject's renal function; and (b) administering ulotaront to the subject if the subject has a creatinine clearance rate greater than or equal to 30 mL/min or an eGFR greater than or equal to 30 mL/min/1.73 m².

In another embodiment, the disclosure provides a method of administering ulotaront to a human subject in need thereof, wherein the subject is on ulotaront with declining renal function, comprising: (a) measuring or evaluating the subject's renal function; (b) administering ulotaront while the subject has a creatinine clearance rate greater than or equal to 30 mL/min or an eGFR greater than or equal to 30 mL/min/1.73 m²; and (c) upon the subject having a creatinine clearance rate less than 30 mL/min, or an eGFR less 30 mL/min/1.73 m², or kidney failure, not administering the ulotaront.

In another embodiment, the disclosure provides a method of administering ulotaront to a human subject in need thereof, wherein the ulotaront is accompanied by written instructions instructing non-administration of ulotaront in the event of a creatinine clearance rate less than a written threshold, or kidney failure, comprising: (a) measuring or evaluating the subject's renal function; and (b) in response to the measurement or evaluation of renal function, based on the written instructions: (i) if the subject has a creatinine clearance rate greater than or equal to 30 mL/min or an eGFR greater than or equal to 30 mL/min/1.73 m², administering ulotaront; (ii) if the subject has a creatinine clearance rate less than 30 mL/min, or an eGFR less than 30 mL/min/1.73 m², or has kidney failure, not administering ulotaront.

In another embodiment, the disclosure provides a method of reducing exposure to SEP-103 during chronic administration of ulotaront, in a human subject in need thereof, comprising: (a) measuring or evaluating the subject's renal function; (b) administering ulotaront to the subject if the subject has a renal function above a threshold; and (c) not administering ulotaront to the subject if the subject has a renal function below the threshold.

In another embodiment, the disclosure provides a method of reducing exposure to SEP-103 during chronic administration of ulotaront, in a human subject in need thereof with renal impairment, comprising: (a) measuring or evaluating the subject's renal function; (b) administering ulotaront to the subject if the subject has a renal function above a threshold; and (c) not administering ulotaront to the subject if the subject has a renal function below the threshold.

In another embodiment, the disclosure provides a method of administering ulotaront to a human subject in need thereof, wherein the ulotaront is accompanied by written instructions instructing non-administration of ulotaront if the subject has a renal function below a threshold, comprising: (a) measuring or evaluating the subject's renal function; and (b) providing in response to the measurement or evaluation of renal function, based on the written instructions: (i) administering ulotaront to the subject if the subject has a renal function above the threshold; and (ii) not administering ulotaront to the subject if the subject has a renal function below the threshold.

In another embodiment, the disclosure provides a method of administering ulotaront to a human subject in need thereof, wherein the subject is on ulotaront administration with declining renal function, comprising: (a) measuring or evaluating the subject's renal function; (b) administering ulotaront to the subject while the subject has a renal function above a threshold; and (c) not administering ulotaront if the subject has a renal function below the threshold.

Additional advantages of the disclosure are set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE FIGURES

The accompanying drawings, which are incorporated in and constitute a part of this specification, illustrate several embodiments of the disclosure and together with the description serve to explain the principles of the disclosure.

FIG. 1 is a scatter plot of ratios of exposure of SEP-103 to SEP-856 in individual subjects, relative to creatine clearance rates, obtained from three different human clinical studies, as described in Example 2.

FIG. 2 is a graph plotting mean SEP-856 plasma concentrations over time in healthy subjects (circles), subjects with mild renal impairment (triangles), and subjects with moderate renal impairment (squares), from the study described in Example 3.

FIG. 3 is a graph plotting mean SEP-103 plasma concentrations over time in healthy subjects (circles), subjects with mild renal impairment (triangles), and subjects with moderate renal impairment (squares), from the study described in Example 3.

FIG. 4 is a graph plotting individual and geometric mean SEP-103 MR_{AUC} (metabolite-to-parent ratio based on AUCO_{0-∞}) by renal function group (PK Population) in healthy subjects, subjects with mild renal impairment, and subjects with moderate renal impairment, from the study described in Example 3.

FIG. 5 is a graph plotting individual and geometric mean SEP-103 MR_{Cmax} (metabolite-to-parent ratio based on Cₘₐₓ) by renal function group (PK Population) in healthy subjects, subjects with mild renal impairment, and subjects with moderate renal impairment, from the study described in Example 3.

### DETAILED DESCRIPTION

All published documents cited herein are hereby incorporated herein by reference in their entirety.

### Use of Terms

As used herein, the singular forms “a,” “an” and “the” are intended to include the plural forms as well, unless the context clearly indicates otherwise.

Unless otherwise specified, the word “includes” (or any variation thereon, e.g., “include,” “including,” etc.) is intended to be open-ended. For example, “A includes 1, 2 and 3” means that A includes, but is not limited to, 1, 2 and 3.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

When ranges are given by specifying the lower end of a range separately from the upper end of the range, or particular numerical values are specified, it will be understood that a range can be defined by selectively combining any of the lower end variables, upper end variables, and particular numerical values that is mathematically possible. When ranges are stated as extending from one endpoint to another endpoint, it will be understood that the two endpoints are included in the range. However, it will also be understood that a from/to range also includes an embodiment in which the range is defined as between the two specified endpoints, and that the term “between” can be substituted for the “from/to” language to omit the endpoints from the range.

The present disclosure describes various embodiments. A person of ordinary skill In the art reviewing the disclosure will readily recognize that various embodiments can be combined in any variation. For example, embodiments of the disclosure include treatment of various disorders, patient populations, administrations of dosage forms, at various dosages, minimization of various adverse events, and improvements in various efficacy measures, etc. Any combinations of various embodiments are within the scope of the disclosure.

When published test methodologies and diagnostic instruments are referred to herein, it will be understood that the test methodology or diagnostic instrument is performed based on the version in effect on Jan. 1, 2023, unless otherwise stated to the contrary herein. This is true even when the methodology or instrument is defined herein based on a publication reporting an earlier version.

Unless defined otherwise, all technical and scientific terms used herein have the same meaning as commonly understood by one of ordinary skill in the art to which this disclosure belongs.

### Definitions

As used herein, “administering” or “administration” of ulotaront encompasses ingestion or swallowing of the ulotaront, administration of the ulotaront to a human subject by a third party such as a nurse practitioner, the delivery of ulotaront by a pharmacist, or the prescription of ulotaront by a physician or other licensed prescriber. The administering can be for ulotaront, or a pharmaceutically acceptable salt thereof, or a prodrug or other pharmaceutically acceptable derivative thereof, using any suitable formulation or route of administration, e.g., as described herein.

In many of the embodiments of the disclosure, ulotaront is said to be administered depending on whether one or more conditions is satisfied. Each of these embodiments contemplates additional embodiments in which ulotaront is only administered if the one or more conditions is satisfied.

An “AE” or “adverse event” is any untoward medical occurrence associated with the use of a drug in humans, whether or not considered drug related. Those untoward medical occurrences that occur after first administration of study drug are considered AEs. An AE can, therefore, be any unfavorable and unintended sign (including an abnormal laboratory finding), symptom, or disease occurring after the administration of a medicinal (investigational) product, whether or not considered related to the medicinal (investigational) product. AEs may include the onset of new illness and the exacerbation of pre-existing conditions.

As used herein, an “at risk” individual is an individual who is at risk of developing a disorder to be treated or an adverse event to be prevented. This may be shown, for example, by one or more risk factors, which are measurable parameters that correlate with development of a disorder and are known in the art. When a method is said to treat a condition without causing or eliciting an adverse event, it will be understood that the methods can be performed in patients at risk of the adverse event.

The term “neuropsychiatric medication” refers to any medication having neuropsychiatric activity. One class of neuropsychiatric medications is the typical and atypical antipsychotics. Thus, whenever the term “neuropsychiatric medication” is employed herein, the term “typical or atypical antipsychotic” can be substituted therefor. A “neuropsychiatric therapy” is therapy based on administering one or more neuropsychiatric medications.

“Pharmaceutically acceptable” or “physiologically acceptable” refers to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of ulotaront include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions (X) are quite acceptable as synthetic intermediates. Thus, X may be pharmaceutically undesirable anions, such as iodide, oxalate, trifluoromethanesulfonate and the like, when such salts are chemical intermediates.

As used herein, the term “pharmaceutically acceptable excipient” includes, without limitation, any binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, disintegrants, lubricant, polymer matrix system, and polishing agents, which has been approved by or is otherwise acceptable to the United States Food and Drug Administration upon proper qualification for use in humans or domestic animals.

As used herein, “prevention” or “preventing” refers to a regimen that protects against the onset of the disorder such that the clinical symptoms of the disorder do not develop. Accordingly, “prevention” relates to administration of a therapy to a subject before signs of the diseases are detectable in the subject (for example, administration of a therapy in the absence of a detectable symptom of the disorder). The subject may be an individual at risk of developing the disorder.

As used herein, the term “renal impairment” is used as the term is used in U.S. FDA's Guidance for Industry Pharmacokinetics in Patients with Impaired Renal Function-Study Design, Data Analysis, and Impact on Dosing (Draft Guidance September 2020). As explained in that document, there are different ways to assess renal function. Measurement of the glomerular filtration rate (GFR) using exogenous markers such as inulin, iothalamate, EDTA, diethylene triamine pentaacetic acid, and iohexol provides a more accurate assessment of renal function than estimating equations. In addition, measured creatinine clearance (CLcr) using timed urine samples is sometimes used to assess renal function. However, these methods are not routinely used in clinical practice.

Serum creatinine-based equations include:

(1) Estimated GFR (eGFR) calculated using a contemporary, widely accepted equation for the population being studied. In clinical practice, eGFR values are standardized to a body surface area (BSA) value of 1.73 m² and expressed and reported in units of mL/min/1.73 m². Renal clearance of a drug is proportional to individual GFR (expressed as mL/min) and not BSA-standardized GFR, hence BSA-standardization will not be appropriate in patients with BSAs different than the standard (1.73 m²). To individualize GFR for drug dosing, multiply the standardized GFR by the individual's BSA calculated using an appropriate formula and divide by 1.73.
(2) Estimated creatinine clearance (CLcr) in mL/min calculated using the Cockcroft-198 Gault (C-G) equation. In overweight or obese individuals, use of alternative body weight metrics such as ideal body weight (IBW) or adjusted body weight (ABW) when calculating CLcr is likely to provide a more accurate estimate of renal function than total body weight.

The document also includes the following useful table for definitional purposes in this disclosure:

### Classifications of Renal Function^{a,b} for Dedicated Renal Impairment Studies

| | Range of Values for |
|---|---|
| Description | Renal Function (mL/min) |
| Control (normal renal function) | ≥90 |
| Mild impairment | 60-89 |
| Moderate impairment | 30-59 |
| Severe impairment | 15-29 |
| Kidney failure^{c} | <15 or dialysis patients |
| | on non-dialysis days |

^{a}eGFR: estimate of GFR based on an estimation equation and expressed in mL/min. To convert mL/min/1.73 m² to mL/min multiply by the individual's BSA calculated using an appropriate formula and divide by 1.73.
^{b}CLcr: estimated creatinine clearance based on the C-G equation.
^{c}Kidney failure: This classification is strictly for the purposes of conducting a dedicated renal impairment study and 243 should not be used for the purposes of classifying kidney disease.

Note further that patients on dialysis are commonly referred to as patients with end stage renal disease or “ESRD.” For purposes of this disclosure, when a cutoff is expressed herein based on kidney failure it will be understood that the cutoff can also be expressed based on ESRD and vice versa. In addition, for purposes of this disclosure, when a cutoff is expressed herein based on creatinine clearance, it can alternatively be expressed as eGFR simply by substituting the unit of measure (mL/min/1.73 m²) for (mL/min), and vice versa.

The term “selecting” refers to the act of choosing from a number or group by fitness or preference. In the context of the current disclosure, ulotaront is selected from a group of generally recognized neuropsychiatric medications, for the treatment of any of the neuropsychiatric conditions described herein.

As used herein, the term SEP-103, SEP-383103, or SEP-383-103, refers to a metabolite of ulotaront formed in vivo when ulotaront is administered to humans. The metabolite has the following chemical structure:

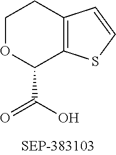 [SMILES: O=C(O)[C@@H]1OCCc2ccsc21]

As used herein, the term “significantly” refers to a level of statistical significance. The level of statistical significance can be p<0.1, p<0.05, p<0.01, p<0.005, or p<0.001. Unless otherwise specified, the level of statistical significance when the term “significant,” “significantly,” or other variations of the term are used is p<0.05. When a measurable result or effect is expressed or identified herein, it will be understood that the result or effect is preferably evaluated based upon its statistical significance relative to a baseline such as placebo. In like manner, when a treatment or benefit is described herein, it will be understood that the treatment or benefit preferably shows efficacy in a population of patients to a degree of statistical significance.

As used herein, “subject” or “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, “tapering” refers to the period during which a subject is transitioned from a neuropsychiatric medication to ulotaront in the methods of this disclosure and is coterminous with the period of time during which the neuropsychiatric medication and ulotaront are co-administered. The term is also used when referring to ulotaront doses administered during the tapering period (i.e., first ulotaront dose) and neuropsychiatric medication doses administered during the tapering period (i.e., first NM dose). When used to describe a dose, no implication should be drawn from the use of the term tapering as to whether the ulotaront dose is being increased or decreased or staying the same during the tapering period, or whether the neuropsychiatric medication dose is being increased or decreased or staying the same.

As used herein, the term “therapeutically effective” or “effective” refers to an amount or dose that is effective to elicit the desired biological or medical response, including the amount of a compound that, when administered to a subject for treating a disorder, is sufficient to accomplish such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). The pharmacokinetics (PK) characteristics for both single and multiple dose administration of SEP-363856 were consistent, with median time of the maximum observed plasma concentration (Tₘₐₓ) ranging between 1.25 and 4 hours and 2 and 4 hours for SEP-363856. An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound. A dose given during a “therapeutically effective period” is by definition a “therapeutically effective amount.”

As used herein, the terms “treatment,” “treat,” and “treating” refer to reversing, alleviating, delaying the onset of, or inhibiting the progress of a disease or disorder, or one or more symptoms thereof, including but not limited to therapeutic benefit. In some embodiments, treatment is administered after one or more symptoms have developed, for example, acute exacerbation of symptoms. In some embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example to prevent or delay their recurrence.

Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder.

In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

“Ulotaront” (a/k/a SEP-363856, SEP-363856, SEP-856), as referred to herein for use in the methods of the present disclosure, has the chemical name(S)-(4,5-dihydro-7H-thieno[2,3-c]pyran-7-yl)-N-methylmethanamine (which may be abbreviated as “(S)-TPMA”). Ulotaront has the following structure:

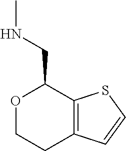

Unless stated otherwise, or unless context requires otherwise, for purposes of this disclosure, the term “ulotaront,” standing alone, includes the free form of ulotaront and also includes its pharmaceutically acceptable salts, hydrates, solvates, amorphous and crystalline forms. When the free form is intended, or any other form or salt is specifically intended, it will be stated as such expressly.

Ulotaront can be used in the methods described herein as the free base or in the form of a pharmaceutically acceptable salt. In preferred embodiments, a hydrochloric acid (HCl) salt of ulotaront is used in the methods described herein. Ulotaront, or a pharmaceutically acceptable salt thereof, including its HCl crystalline forms, can be obtained according to the production methods described in PCT Patent Publication No. WO2011/069063 (U.S. Pat. No. 8,710,245, issued Apr. 29, 2014) or PCT Patent Publication No. WO2019/161238, which are incorporated herein by reference in their entirety and for all purposes, or a method analogous thereto.

Also provided herein are pharmaceutical compositions and dosage forms, comprising ulotaront, or a pharmaceutically acceptable salt thereof, and one or more pharmaceutically acceptable excipients. Compositions and dosage forms provided herein may further comprise one or more additional active ingredients. Ulotaront as described herein may be administered as part of a pharmaceutical composition.

### DISCUSSION

Accordingly, in one embodiment, the disclosure provides a method of administering ulotaront to a human subject in need thereof with renal impairment, comprising: (a) measuring or evaluating the subject's renal function; and (b) administering ulotaront to the subject if the subject has a creatinine clearance rate greater than or equal to 30 mL/min.

In another embodiment, the disclosure provides a method of administering ulotaront to a human subject in need thereof, wherein the subject is treatment naïve or on a preexisting regimen of neuropsychiatric therapy, comprising: (a) measuring or evaluating the subject's renal function; and (b) administering ulotaront to the subject if the subject has a creatinine clearance rate greater than or equal to 30 mL/min.

In another embodiment, the disclosure provides a method of administering ulotaront to a human subject in need thereof, wherein the subject is on ulotaront with declining renal function, comprising: (a) measuring or evaluating the subject's renal function; (b) administering ulotaront while the subject has a creatinine clearance rate greater than or equal to 30 mL/min; and (c) upon the subject having a creatinine clearance rate less than 30 mL/min, or kidney failure, not administering the ulotaront.

In another embodiment, the disclosure provides a method of administering ulotaront to a human subject in need thereof, wherein the ulotaront is accompanied by written instructions instructing non-administration of ulotaront in the event of a creatinine clearance rate less than a written threshold, or kidney failure, comprising: (a) measuring or evaluating the subject's renal function; and (b) in response to the measurement or evaluation of renal function, based on the written instructions: (i) if the subject has a creatinine clearance rate greater than or equal to 30 mL/min, administering ulotaront; (ii) if the subject has a creatinine clearance rate less than 30 mL/min, or has kidney failure, not administering ulotaront.

In another embodiment, the disclosure provides a method of reducing exposure to SEP-103 during chronic administration of ulotaront, in a human subject in need thereof, comprising: (a) measuring or evaluating the subject's renal function; (b) administering ulotaront to the subject if the subject has a renal function above a threshold; and (c) not administering ulotaront to the subject if the subject has a renal function below the threshold.

In another embodiment, the disclosure provides a method of reducing exposure to SEP-103 during chronic administration of ulotaront, in a human subject in need thereof with renal impairment, comprising: (a) measuring or evaluating the subject's renal function; (b) administering ulotaront to the subject if the subject has a renal function above a threshold; and (c) not administering ulotaront to the subject if the subject has a renal function below the threshold.

In another embodiment, the disclosure provides a method of administering ulotaront to a human subject in need thereof, wherein the ulotaront is accompanied by written instructions instructing non-administration of ulotaront if the subject has a renal function below a threshold, comprising: (a) measuring or evaluating the subject's renal function; and (b) providing in response to the measurement or evaluation of renal function, based on the written instructions: (i) administering ulotaront to the subject if the subject has a renal function above the threshold; and (ii) not administering ulotaront to the subject if the subject has a renal function below the threshold.

In another embodiment, the disclosure provides a method of administering ulotaront to a human subject in need thereof, wherein the subject is on ulotaront administration with declining renal function, comprising: (a) measuring or evaluating the subject's renal function; (b) administering ulotaront to the subject while the subject has a renal function above a threshold; and (c) not administering ulotaront if the subject has a renal function below the threshold.

Any of the embodiments of this disclosure can be practiced to prevent accumulation of SEP-103 in the body exceeding levels studied and deemed safe in preclinical toxicology studies. In some embodiments, the methods of this disclosure, and the thresholds/cutoffs for when ulotaront is administered or not administered, prevent blood concentrations of SEP-103 from exceeding concentrations studied and deemed to be safe in preclinical toxicology studies (i.e., 700 ng/mL in rats and 1100 ng/ml in dogs). In other embodiments, the methods of this disclosure, and the thresholds/cutoffs for when ulotaront is administered or not administered, prevent exposure to SEP-103 from exceeding levels studied and deemed to be safe in preclinical toxicology studies (i.e., 11700 ng*hr/mL in rats and 12200 ng*hr/mL in dogs).

In some embodiments, ulotaront is administered if the subject has a creatinine clearance rate greater than or equal to 30 mL/min or an eGFR greater than or equal to 30 mL/min/1.73 m². Expressed another way, ulotaront is administered if the subject is non-renally impaired or has mild or moderate renal impairment. I.e., the threshold for ulotaront administration is a creatinine clearance rate greater than or equal to 30 mL/min or an eGFR greater than or equal to 30 mL/min/1.73 m² or the upper bound of severe renal impairment. In these embodiments, ulotaront is not administered if the subject has a creatinine clearance rate less than 30 mL/min or an eGFR less than 30 mL/min/1.73 m², or the subject has severe renal impairment or is with kidney failure.

In some embodiments, ulotaront is administered if the subject has a creatinine clearance rate greater than or equal to 45 mL/min or an eGFR greater than or equal to 45 mL/min/1.73 m². Expressed another way, ulotaront is administered if the subject is non-renally impaired or has mild renal impairment or moderate renal impairment with CLcr or eGFR greater than or equal to 45 mL/min or greater than or equal to 45 mL/min/1.73 m². I.e., the threshold for ulotaront administration is a creatinine clearance rate greater than or equal to 45 mL/min or an eGFR greater than or equal to 45 mL/min/1.73 m². In these embodiments, ulotaront is not administered if the subject has a creatinine clearance rate less than 45 mL/min or an eGFR less than 45 mL/min/1.73 m², or the subject has moderate renal impairment with CLcr or eGFR less than 45 mL/min or 45 mL/min/1.73 m², severe renal impairment, or is with kidney failure.

In some embodiments, ulotaront is administered if the subject has a creatinine clearance rate greater than or equal to 60 mL/min or an eGFR greater than or equal to 60 mL/min/1.73 m². Expressed another way, ulotaront is administered if the subject is non-renally impaired or has mild renal impairment. I.e., the threshold for ulotaront administration is a creatinine clearance rate greater than or equal to 60 mL/min or an eGFR greater than or equal to 60 mL/min/1.73 m² or the upper bound of moderate renal impairment. In these embodiments, ulotaront is not administered if the subject has a creatinine clearance rate less than 60 mL/min or an eGFR less than 60 mL/min/1.73 m², or the subject has moderate or severe renal impairment or is with kidney failure.

In some embodiments, ulotaront is administered if the subject has a creatinine clearance rate greater than or equal to 90 mL/min or an eGFR greater than or equal to 90 mL/min/1.73 m². Expressed another way, ulotaront is administered if the subject is non-renally impaired. I.e., the threshold for ulotaront administration is a creatinine clearance rate greater than or equal to 90 mL/min or an eGFR greater than or equal to 90 mL/min/1.73 m² or the upper bound of mild renal impairment. In these embodiments, ulotaront is not administered if the subject has a creatinine clearance rate less than 90 mL/min or an eGFR less than 90 mL/min/1.73 m², or the subject has mild or moderate or severe renal impairment or is with kidney failure.

In some embodiments, ulotaront is administered if the subject has a creatinine clearance rate greater than or equal to 15 mL/min or an eGFR greater than or equal to 15 mL/min/1.73 m², and does not have ESRD. Expressed another way, ulotaront is administered if the subject is non-renally impaired or has mild or moderate or severe renal impairment. I.e., the threshold for ulotaront administration is a creatinine clearance rate greater than or equal to 15 mL/min or an eGFR greater than or equal to 15 mL/min/1.73 m² or the upper bound of kidney failure. In these embodiments, ulotaront is not administered if the subject has a creatinine clearance rate less than 15 mL/min or an eGFR less than 15 mL/min/1.73 m² or ESRD.

In various embodiments, the subject is described based on the subject's degree of renal impairment or non-renal impairment. Thus, in some embodiments, the subject is not renally impaired. In some embodiments, the subject is renally impaired. In some embodiments, the subject is mildly, moderately, or severely renally impaired, or has kidney failure. In some embodiments, the subject is mildly renally impaired. In some embodiments the subject is moderately renally impaired. In some embodiments, the subject is severely renally impaired. In some embodiments the subject has kidney failure.

In any of the embodiments of the current disclosure, except those based on a threshold at the upper bound of mild renal impairment, the subject can be renally impaired, and is preferably renally impaired since the prescribing physician will need to evaluate the subject's suitability for ulotaront administration based on the applicable threshold of renal impairment.

In other embodiments, particularly suitable when a subject has declining renal function, the subject will initially have renal impairment above the threshold and the subject's renal function will be measured periodically a plurality of times, and the ulotaront administration is ceased when the subject's renal function falls below the threshold.

All of the embodiments of the current disclosure also contemplate the potential for administration of an alternative neuropsychiatric medication, suitable for treating the condition for which ulotaront is under consideration, in the event that ulotaront is not administered. The main requirement for the alternative medication is that it be suitable for administration to subjects with renal function below the threshold at which ulotaront is not administered, and that it be suitable for treatment of the condition suffered by the subject. The alternative medication can itself be subject to restrictions on its administration to renally impaired subjects, such as requirements for dose reduction, as long as some level of dosing is suitable for subjects having renal function below the applicable threshold.

Thus, in some embodiments, the methods of the current disclosure further comprise providing an alternative neuropsychiatric medication suitable for administration to human subjects with renal function below the applicable threshold. In some embodiments, the applicable threshold is 15 mL/min CLcr or 15 ml/min/1.73 m² eGFR or the upper bound of kidney failure. In some embodiments, the applicable threshold is 30 mL/min CLcr or 30 ml/min/1.73 m² eGFR or the upper bound of severe renal impairment. In some embodiments, the applicable threshold is 45 mL/min CLcr or 45 ml/min/1.73 m² eGFR. In some embodiments, the applicable threshold is 60 mL/min CLcr or 60 ml/min/1.73 m² eGFR or the upper bound of moderate renal impairment. In some embodiments, the applicable threshold is 90 mL/min CLcr or 90 ml/min/1.73 m² eGFR or the upper bound of mild renal impairment.

All of the embodiments of the current disclosure also contemplate written instructions accompanying the ulotaront, defining a threshold of renal function at which ulotaront is contraindicated or is not to be administered, and instructing the user not to administer (i.e., use, ingest, prescribe or otherwise provide) ulotaront if the subject under treatment has a renal function falling below the applicable threshold. The written instructions can be actively provided, as when written instructions are furnished by the pharmacist to the subject. Alternatively, the written instructions can passively accompany the ulotaront or be passively provided, as when ulotaront is prescribed by a physician or physically delivered to a subject by a pharmacist without written instructions, but written instructions for the ulotaront, generated by the actual manufacturer of the ulotaront that is administered, are provided on-line through resources such as the manufacturer's web site or U.S. FDA's DailyMeds web site. (https://dailymed.nlm.nih.gov/dailymed/) (last accessed Feb. 18, 2023).

All of the embodiments of the disclosure further contemplate either measuring the subject's renal function by testing the subject or having the subject tested, preferably for creatinine clearance. Thus, the entity or person actually administering the ulotaront need not physically administer the test for renal function, as long as the entity or person orders the test, or otherwise considers the results of such testing during the decision whether to administer ulotaront. Thus, the methods can also be practiced by evaluating a subject's renal function based on test results obtained from the subject, another physician, or a third party testing laboratory.

Thus, in any of the embodiments of the current disclosure, (a) the ulotaront is accompanied by written instructions instructing non-administration of the ulotaront if the subject has renal function below a threshold, and (b) the method further comprises, in response to the measurement or evaluation of renal function, based on the written instructions: (i) administering ulotaront to the subject if the subject has renal function above the threshold; and (ii) not administering ulotaront to the subject if the subject has renal function below the threshold. In some embodiments, the applicable threshold is 15 mL/min CLcr or 15 ml/min/1.73 m² eGFR or the upper bound of kidney failure. In some embodiments, the applicable threshold is 30 mL/min CLcr or 30 ml/min/1.73 m² eGFR or the upper bound of severe renal impairment. In some embodiments, the applicable threshold is 45 mL/min CLcr or 45 ml/min/1.73 m² eGFR. In some embodiments, the applicable threshold is 60 mL/min CLcr or 60 ml/min/1.73 m² eGFR or the upper bound of moderate renal impairment. In some embodiments, the applicable threshold is 90 mL/min CLcr or 90 ml/min/1.73 m² eGFR or the upper bound of mild renal impairment.

In some embodiments, the subject is treatment naïve and under consideration for ulotaront administration. In other embodiments, the subject is on a preexisting regimen of a neuropsychiatric therapy and under consideration for ulotaront administration. The subject need not go off the preexisting therapy entirely when ulotaront is administered. Thus, in some embodiments, the subject is on a preexisting regimen of a neuropsychiatric therapy and, when the ulotaront is administered, it is administered in addition to the preexisting regimen. In other embodiments, the subject is on a preexisting regimen of a neuropsychiatric therapy and, when the ulotaront is administered, it is administered instead of the preexisting regimen.

The transition from a preexisting regime to ulotaront can also involve a tapering period. Thus, in some embodiments, the subject is on a preexisting regimen of neuropsychiatric therapy, and the method further comprises administering the preexisting regimen and the ulotaront during a tapering period. Although not critical, the tapering period typically lasts from one week to six months, preferably from two weeks to eight weeks. In other embodiments, the preexisting regimen of neuropsychiatric therapy comprises a treatment dose of a neuropsychiatric medication during a first treatment period, and the method further comprises: (a) after the first treatment period, administering a tapering dose of the neuropsychiatric medication and a first subtherapeutic dose of ulotaront during a tapering period; and (c) after the tapering period, administering a final therapeutically effective dose of the ulotaront without administering the neuropsychiatric medication

The initiation of ulotaront administration can also involve a titration scheme. Thus, in still further embodiments the ulotaront is titrated to a final therapeutically effective dose over a period of from one week to six months, preferably from two weeks to three months starting with a dose of 50% or less than the final therapeutically effective dose.

In other embodiments, the subject is on a preexisting regimen of a neuropsychiatric therapy, and the preexisting regimen produces an inadequate therapeutic response or a clinically significant adverse event. In more particular embodiments, the preexisting regimen produces a clinically significant adverse event selected from akathisia, blood prolactin abnormal, blood prolactin increased, blood triglycerides increased, body mass index increased, bradykinesia, bruxism, cogwheel rigidity, dermatillomania, diabetes mellitus, drooling, dyskinesia, dyslipidaemia, dyssomnia, dystonia, electrocardiogram QT prolonged, enuresis, excessive eye blinking, extrapyramidal disorder, galactorrhoea, glucose tolerance impaired, glycosuria, hyperkinesia, hyperprolactinaemia, impaired fasting glucose, increased appetite, metabolic syndrome, muscle rigidity, nuchal rigidity, obesity, obsessive-compulsive disorder, oculogyric crisis, oromandibular dystonia, orthostatic hypertension, overweight, pancreatitis chronic, parkinsonian gait, parkinsonism, psychomotor retardation, restless legs syndrome, restlessness, salivary hypersecretion, sedation, sexual dysfunction, tardive dyskinesia, tic, tongue biting, tongue spasm, torticollis, type 2 diabetes mellitus, and weight increase.

In any of the embodiments of the disclosure, it will be understood that the patient has a condition suitable for treatment by ulotaront. A disclosed by Dedic 2019 and Koblan 2020, and as further supported by phase II/III clinical trial protocols posted on clinicaltrials.gov (last accessed Feb. 18, 2023), ulotaront has a broad psychiatric profile and behavioral signatures relevant to a variety of mental disorders or conditions.

Thus, in some embodiments, the subject has a condition selected from schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, aggression, delirium, Parkinson's psychosis, excitative psychosis, Tourette's syndrome, organic or NOS psychosis, seizure, agitation, post-traumatic stress disorder, behavior disorder, neurodegenerative disease, Alzheimer's disease, Parkinson's disease, dyskinesias, Huntington's disease, dementia, mood disorder, anxiety, affective disorders (e.g., depression, e.g., major depressive disorder and dysthymia; bipolar disorder, e.g., bipolar depressive disorder; manic disorder; seasonal affective disorder; and attention deficit disorder (ADD) and attention deficit hyperactivity disorder (ADHD)), obsessive-compulsive disorder, vertigo, epilepsy, pain (e.g., neuropathic pain, sensitization accompanying neuropathic pain, and inflammatory pain), fibromyalgia, migraine, cognitive impairment, movement disorder, restless leg syndrome (RLS), multiple sclerosis, sleep disorder, sleep apnea, narcolepsy, excessive daytime sleepiness, jet lag, drowsy side effect of medications, insomnia, substance abuse or dependency (e.g., nicotine, cocaine), addiction, eating disorder, sexual dysfunction, hypertension, emesis, Lesche-Nyhane disease, Wilson's disease, autism, Huntington's chorea, and premenstrual dysphoria.

In other embodiments, the subject has a condition selected from psychosis, mood disorders, anxiety disorders, neurocognitive disorders, substance use disorders, and sleep disorders. In other embodiments, the subject has a condition selected from schizophrenia, depression (including adjunctive major depressive disorder or aMDD), and anxiety (including general anxiety disorder or GAD).

In any of the embodiments of the disclosure, the ulotaront is provided or administered in a therapeutically effective dose which, when administered, is the same regardless of the subject's renal function. Thus, in another embodiment, the method further comprises administering ulotaront at a therapeutically effective dose, which is the same regardless of whether the subject is non-renally impaired or impaired above a relevant threshold, and as long as the subject's renal function is above the threshold. In another embodiment, the method further comprises administering ulotaront at a therapeutically effective dose which is the same regardless of whether the subject is non-renally impaired or mildly renally impaired or moderately renally impaired, and as long as the subject is non-renally impaired or mildly renally impaired or moderately renally impaired.

In one embodiment, the ulotaront is administered in a dose of from 10 to 150 or 25 to 100 mg/day of ulotaront or a pharmaceutically acceptable salt thereof. In another embodiment, the ulotaront is administered in a dose selected from 25, 50, 75, 100, 125, and 150 mg/day of ulotaront or a pharmaceutically acceptable salt thereof. In some embodiments, the ulotaront is administered orally. In some embodiments, the ulotaront is administered once daily in the fed or fasted state. In some embodiments, the ulotaront is administered as the free base or as a pharmaceutically acceptable salt. In some embodiments, the ulotaront is administered as the hydrochloride salt.

### ALTERNATIVE/SUPPLEMENTAL EMBODIMENTS

Below are alternative and supplemental descriptions of the various embodiments of the disclosure. Note that, when reference to an Embodiment is made by numerical reference, it is intended to refer to all embodiments encompassed by that number. Thus, for example, when reference to Embodiment 1 is made, it is intended also to refer to Embodiment 1a and 1b.

Embodiment 1. A method of administering ulotaront to a human subject in need thereof with renal impairment, comprising: (a) measuring or evaluating the subject's renal function; and (b) administering ulotaront to the subject if the subject has a creatinine clearance rate greater than or equal to 30 mL/min or an eGFR greater than or equal to 30 mL/min/1.73 m².

Embodiment 1a. A method of administering ulotaront to a human subject in need thereof with renal impairment, comprising: (a) measuring or evaluating the subject's renal function; (b) administering ulotaront to the subject if the subject has a creatinine clearance rate greater than or equal to 30 mL/min or an eGFR greater than or equal to 30 mL/min/1.73 m²; and (c) not administering ulotaront if the subject has a creatinine clearance rate less than 30 mL/min, or an eGFR less than 30 mL/min/1.73 m², or has kidney failure.

Embodiment 1b. A method of administering ulotaront to a human subject in need thereof, comprising: (a) measuring or evaluating the subject's renal function; and (b) administering ulotaront to the subject if the subject has a creatinine clearance rate greater than or equal to 30 mL/min or an eGFR greater than or equal to 30 mL/min/1.73 m².

Embodiment 1c. A method of administering ulotaront to a human subject in need thereof with renal impairment, comprising: (a) measuring or evaluating the subject's renal function; (b) administering ulotaront to the subject if the subject has a creatinine clearance rate greater than or equal to 30 mL/min or an eGFR greater than or equal to 30 mL/min/1.73 m²; and (c) not administering ulotaront if the subject has a creatinine clearance rate less than 30 mL/min, or an eGFR less than 30 mL/min/1.73 m², or has kidney failure.

Embodiment 2. A method of administering ulotaront to a human subject in need thereof, wherein the subject is treatment naïve or on a preexisting regimen of neuropsychiatric therapy, comprising: (a) measuring or evaluating the subject's renal function; and (b) administering ulotaront to the subject if the subject has a creatinine clearance rate greater than or equal to 30 mL/min or an eGFR greater than or equal to 30 mL/min/1.73 m².

Embodiment 2a. A method of administering ulotaront to a human subject in need thereof, wherein the subject is (i) treatment naïve or on a preexisting regimen of neuropsychiatric therapy, and (ii) under consideration for ulotaront administration, comprising: (a) measuring or evaluating the subject's renal function; (b) administering ulotaront to the subject if the subject has a creatinine clearance rate greater than or equal to 30 mL/min or an eGFR greater than or equal to 30 mL/min/1.73 m²; and (c) not administering ulotaront if the subject has a creatinine clearance rate less than 30 mL/min, or an eGFR less than 30 mL/min/1.73 m², or has kidney failure.

Embodiment 3. A method of administering ulotaront to a human subject in need thereof, wherein the subject is on ulotaront with declining renal function, comprising: (a) measuring or evaluating the subject's renal function; (b) administering ulotaront while the subject has a creatinine clearance rate greater than or equal to 30 mL/min or an eGFR greater than or equal to 30 mL/min/1.73 m²; and (c) upon the subject having a creatinine clearance rate less than 30 mL/min, or an eGFR less than 30 mL/min/1.73 m², or having kidney failure, not administering the ulotaront.

Embodiment 4. A method of administering ulotaront to a human subject in need thereof, wherein the ulotaront is accompanied by written instructions instructing non-administration of ulotaront in the event of a creatinine clearance rate less than 30 mL/min, or an eGFR less than 30 mL/min/1.73 m², or kidney failure, comprising: (a) measuring or evaluating the subject's renal function; and (b) in response to the measurement or evaluation of renal function, based on the written instructions: (i) if the subject has a creatinine clearance rate greater than or equal to 30 mL/min or an eGFR greater than or equal to 30 mL/min/1.73 m², administering ulotaront; (ii) if the subject has a creatinine clearance rate less than 30 mL/min, or an eGFR less than 30 mL/min/1.73 m², or has kidney failure, not administering ulotaront.

Embodiment 5. A method of reducing exposure to SEP-103 during chronic administration of ulotaront, in a human subject in need thereof, comprising: (a) measuring or evaluating the subject's renal function; (b) administering ulotaront to the subject if the subject has a renal function above a threshold; and (c) not administering ulotaront to the subject if the subject has a renal function below the threshold.

Embodiment 5a. A method of reducing exposure to SEP-103 during chronic administration of ulotaront, in a human subject in need thereof, comprising: (a) measuring or evaluating the subject's renal function; and (b) administering ulotaront to the subject if the subject has a renal function above a threshold.

Embodiment 5b. A method of administering ulotaront to a human subject in need thereof, comprising: (a) measuring or evaluating the subject's renal function; (b) administering ulotaront to the subject if the subject has a renal function above a threshold; and (c) not administering ulotaront to the subject if the subject has a renal function below the threshold.

Embodiment 5c. A method of administering ulotaront to a human subject in need thereof, comprising: (a) measuring or evaluating the subject's renal function; and (b) administering ulotaront to the subject if the subject has a renal function above a threshold.

Embodiment 6. A method of reducing exposure to SEP-103 during chronic administration of ulotaront, in a human subject in need thereof with renal impairment, comprising: (a) measuring or evaluating the subject's renal function; (b) administering ulotaront to the subject if the subject has a renal function above a threshold; and (c) not administering ulotaront to the subject if the subject has a renal function below the threshold.

Embodiment 6a. A method of reducing exposure to SEP-103 during chronic administration of ulotaront, in a human subject in need thereof with renal impairment, comprising: (a) measuring or evaluating the subject's renal function; and (b) administering ulotaront to the subject if the subject has a renal function above a threshold.

Embodiment 6b. A method of administering ulotaront to a human subject in need thereof with renal impairment, comprising: (a) measuring or evaluating the subject's renal function; (b) administering ulotaront to the subject if the subject has a renal function above a threshold; and (c) not administering ulotaront to the subject if the subject has a renal function below the threshold.

Embodiment 6c. A method of administering ulotaront to a human subject in need thereof with renal impairment, comprising: (a) measuring or evaluating the subject's renal function; and (b) administering ulotaront to the subject if the subject has a renal function above a threshold.

Embodiment 7. A method of administering ulotaront to a human subject in need thereof, wherein the ulotaront is accompanied by written instructions instructing non-administration of ulotaront if the subject has a renal function below a threshold comprising: (a) measuring or evaluating the subject's renal function; and (b) in response to the measurement or evaluation of renal function, based on the written instructions: (i) administering ulotaront to the subject if the subject has a renal function above the threshold; and (ii) not administering ulotaront to the subject if the subject has a renal function below the threshold.

Embodiment 8. A method of administering ulotaront to a human subject in need thereof, wherein the subject is on ulotaront administration with declining renal function, comprising: (a) measuring or evaluating the subject's renal function; (b) administering ulotaront to the subject while the subject has a renal function above a threshold; and (c) not administering ulotaront if the subject has a renal function below the threshold.

Embodiment 9. The method of any of embodiments 1-8, comprising administering ulotaront to the subject if the subject has a creatinine clearance rate greater than or equal to 30 mL/min or an eGFR greater than or equal to 30 mL/min/1.73 m².

Embodiment 9a. The method of any of embodiments 1-8, comprising administering ulotaront to the subject if the subject has a creatinine clearance rate greater than or equal to 30 mL/min or an eGFR greater than or equal to 30 mL/min/1.73 m², and not administering ulotaront to the subject if the subject has a creatine clearance rate less than 30 mL/min or an eGFR less than 30 mL/min/1.73 m².

Embodiment 9b. The method of any of embodiments 1-8, comprising administering ulotaront to the subject if the subject has a creatinine clearance rate greater than or equal to 45 mL/min or an eGFR greater than or equal to 45 mL/min/1.73 m².

Embodiment 9c. The method of any of embodiments 1-8, comprising administering ulotaront to the subject if the subject has a creatinine clearance rate greater than or equal to 45 mL/min or an eGFR greater than or equal to 45 mL/min/1.73 m², and not administering ulotaront to the subject if the subject has a creatine clearance rate less than 45 mL/min or an eGFR less than 45 mL/min/1.73 m².

Embodiment 10. The method of any of embodiments 1-8, comprising administering ulotaront to the subject if the subject has a creatinine clearance rate greater than or equal to 60 mL/min or an eGFR greater than or equal to 60 mL/min/1.73 m².

Embodiment 10b. The method of any of embodiments 1-8, comprising administering ulotaront to the subject if the subject has a creatinine clearance rate greater than or equal to 60 mL/min or an eGFR greater than or equal to 60 mL/min/1.73 m², and not administering ulotaront to the subject if the subject has a creatine clearance rate less than 60 mL/min or an eGFR less than 60 mL/min/1.73 m².

Embodiment 11. The method of any of embodiments 2, 3, 4, 5, 7, 8, 9, and 10, wherein the subject is non-renally impaired.

Embodiment 12. The method of any of embodiments 1-10, wherein the subject is renally impaired.

Embodiment 13. The method of any of embodiments 1-10, wherein the subject is mildly, moderately, or severely renally impaired, or with kidney failure.

Embodiment 14. The method of any of embodiments 1-10, wherein the subject is mildly renally impaired.

Embodiment 15. The method of any of embodiments 1-10, wherein the subject is moderately renally impaired.

Embodiment 16. The method of any of embodiments 1-10, wherein the subject is severely renally impaired.

Embodiment 17. The method of any of embodiments 1-10, wherein the subject has kidney failure.

Embodiment 18. The method of any of embodiments 1-17, wherein the creatinine clearance rate or threshold prevents blood concentrations of SEP-103 from exceeding levels studied and deemed to be safe in preclinical toxicology studies.

Embodiment 19. The method of any of embodiments 1-17, wherein the creatinine clearance rate or threshold prevents exposure to SEP-103 from exceeding levels greater than those studied and deemed to be safe in preclinical toxicology studies.

Embodiment 20. The method of any of embodiments 5-19, wherein the threshold is the upper bound of moderate renal impairment.

Embodiment 21. The method of any of embodiments 5-19, wherein the threshold is the upper bound of severe renal impairment.

Embodiment 22. The method of any of embodiments 5-19, wherein the threshold is the upper bound of kidney failure.

Embodiment 23. The method of any of embodiments 5-19, wherein the threshold is a creatinine clearance rate of 60 mL/min.

Embodiment 24. The method of any of embodiments 5-19, wherein the threshold is a creatinine clearance rate of 45 mL/min.

Embodiment 25. The method of any of embodiments 5-19, wherein the threshold is a creatinine clearance rate of 30 mL/min.

Embodiment 26. The method of any of embodiments 5-19, wherein the threshold is a creatinine clearance rate of 15 mL/min.

Embodiment 27. The method of any of embodiments 5-19, wherein the threshold is an eGFR of 60 mL/min/1.73 m².

Embodiment 28. The method of any of embodiments 5-19, wherein the threshold is an eGFR of 45 mL/min/1.73 m².

Embodiment 29. The method of any of embodiments 5-19, wherein the threshold is an eGFR of 30 mL/min/1.73 m².

Embodiment 30. The method of any of embodiments 5-19, wherein the threshold is an eGFR of 15 mL/min/1.73 m².

Embodiment 31. The method of any of embodiments 5-30, further comprising providing an alternative neuropsychiatric medication suitable for administration to human subjects with renal function below the threshold.

Embodiment 32. The method of any of embodiments 1-4, further comprising providing an alternative neuropsychiatric medication suitable for administration to human subjects with creatinine clearance below 30 mL/min min or eGFR below 30 mL/min/1.73 m².

Embodiment 33. The method of any of embodiments 5-31, further comprising, if ulotaront is not administered, administering an alternative neuropsychiatric medication suitable for administration to human subjects with renal function below the threshold.

Embodiment 34. The method of any of embodiments 1-4, further comprising, if ulotaront is not administered, administering an alternative neuropsychiatric medication suitable for administration to human subjects with creatinine clearance below 30 mL/min or eGFR below 30 mL/min/1.73 m².

Embodiment 35. The method of any of embodiments 31-34, wherein the neuropsychiatric medication and its major metabolites are excreted or metabolized predominantly outside the kidneys.

Embodiment 36. The method of any of embodiments 5-31, wherein the ulotaront is accompanied by written instructions instructing non-administration of the ulotaront if the subject has renal function below the threshold, further comprising, in response to the measurement or evaluation of renal function, based on the written instructions:
(i) administering ulotaront to the subject if the subject has renal function above the threshold; and
(ii) not administering ulotaront to the subject if the subject has renal function below the threshold.

Embodiment 37. The method of any of embodiments 1-4, wherein the ulotaront is accompanied by written instructions instructing non-administration of the ulotaront if the subject has a creatinine clearance rate less than 30 mL/min creatinine clearance or an eGFR less than 30 mL/min/1.73 m², further comprising, in response to the measurement or evaluation of renal function, based on the written instructions:
(i) administering ulotaront to the subject if the subject has a creatinine clearance rate greater than or equal to 30 mL/min or an eGFR greater than or equal to 30 mL/min/1.73 m²; and
(ii) not administering ulotaront to the subject if the subject has a creatine clearance rate less than 30 mL/min or an eGFR less than 30 mL/min/1.73 m².

Embodiment 38. The method of any of embodiments 1-37, wherein the subject is treatment naïve and under consideration for ulotaront administration.

Embodiment 39. The method of any of embodiments 1-38, wherein the subject is on a preexisting regimen of a neuropsychiatric therapy and under consideration for ulotaront administration.

Embodiment 40. The method of any of embodiments 1-39, wherein the subject is on a preexisting regimen of a neuropsychiatric therapy and, when the ulotaront is administered, it is administered in addition to the preexisting regimen.

Embodiment 41. The method of any of embodiments 1-39, wherein the subject is on a preexisting regimen of a neuropsychiatric therapy and, when the ulotaront is administered, it is administered instead of the preexisting regimen.

Embodiment 42. The method of any of embodiments 1-41, wherein the subject is on a preexisting regimen of a neuropsychiatric therapy, and the preexisting regimen produces an inadequate therapeutic response or a clinically significant adverse event.

Embodiment 43. The method of any of embodiments 1-41, wherein the subject is on a preexisting regimen of a neuropsychiatric therapy, wherein the preexisting regimen produces a clinically significant adverse event selected from akathisia, blood prolactin abnormal, blood prolactin increased, blood triglycerides increased, body mass index increased, bradykinesia, bruxism, cogwheel rigidity, dermatillomania, diabetes mellitus, drooling, dyskinesia, dyslipidaemia, dyssomnia, dystonia, electrocardiogram QT prolonged, enuresis, excessive eye blinking, extrapyramidal disorder, galactorrhoea, glucose tolerance impaired, glycosuria, hyperkinesia, hyperprolactinaemia, impaired fasting glucose, increased appetite, metabolic syndrome, muscle rigidity, nuchal rigidity, obesity, obsessive-compulsive disorder, oculogyric crisis, oromandibular dystonia, orthostatic hypertension, overweight, pancreatitis chronic, parkinsonian gait, parkinsonism, psychomotor retardation, restless legs syndrome, restlessness, salivary hypersecretion, sedation, sexual dysfunction, tardive dyskinesia, tic, tongue biting, tongue spasm, torticollis, type 2 diabetes mellitus, and weight increase.

Embodiment 44. The method of any of embodiments 1-43, comprising measuring the subject's renal function by testing the subject or having the subject tested for creatinine clearance.

Embodiment 45. The method of any of embodiments 1-44, wherein the subject has a condition selected from schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, aggression, delirium, Parkinson's psychosis, excitative psychosis, Tourette's syndrome, organic or NOS psychosis, seizure, agitation, post-traumatic stress disorder, behavior disorder, neurodegenerative disease, Alzheimer's disease, Parkinson's disease, dyskinesias, Huntington's disease, dementia, mood disorder, anxiety, affective disorders (e.g., depression, e.g., major depressive disorder and dysthymia; bipolar disorder, e.g., bipolar depressive disorder; manic disorder; seasonal affective disorder; and attention deficit disorder (ADD) and attention deficit hyperactivity disorder (ADHD)), obsessive-compulsive disorder, vertigo, epilepsy, pain (e.g., neuropathic pain, sensitization accompanying neuropathic pain, and inflammatory pain), fibromyalgia, migraine, cognitive impairment, movement disorder, restless leg syndrome (RLS), multiple sclerosis, sleep disorder, sleep apnea, narcolepsy, excessive daytime sleepiness, jet lag, drowsy side effect of medications, insomnia, substance abuse or dependency (e.g., nicotine, cocaine), addiction, eating disorder, sexual dysfunction, hypertension, emesis, Lesche-Nyhane disease, Wilson's disease, autism, Huntington's chorea, and premenstrual dysphoria.

Embodiment 46. The method of any of embodiments 1-44, wherein the subject has a condition selected from psychosis, mood disorders, anxiety disorders, neurocognitive disorders, substance use disorders, and sleep disorders.

Embodiment 47. The method of any of embodiments 1-44, wherein the subject has a condition selected from schizophrenia, depression (including aMDD), and anxiety (including GAD).

Embodiment 48. The method of any of embodiments 1-47, wherein the ulotaront is administered at a therapeutically effective dose.

Embodiment 49. The method of any of embodiments 1-48, further comprising administering or providing the ulotaront as a therapeutically effective dose, which is the same regardless of whether the human subject is non-renally impaired, mildly renally impaired, or moderately renally impaired, and administering ulotaront at the therapeutically effective dose regardless of whether the human subject is non-renally impaired, mildly renally impaired, or moderately renally impaired.

Embodiment 50. The method of any of embodiments 1-49, wherein the ulotaront is administered in a dose of from 10 to 150 or 25 to 100 mg/day of ulotaront or a pharmaceutically acceptable salt thereof.

Embodiment 51. The method of any of embodiments 1-50, wherein the ulotaront is administered in a dose selected from 25, 50, 75, 100, 125, and 150 mg/day of ulotaront or a pharmaceutically acceptable salt thereof.

Embodiment 52. The method of any of embodiments 1-51, wherein the subject is on a preexisting regimen of neuropsychiatric therapy, further comprising administering the preexisting regimen and the ulotaront during a tapering period.

Embodiment 53. The method of any of embodiments 1-52, wherein the ulotaront is titrated to a final therapeutically effective dose over a period of from one week to six months, starting with a dose of 50% or less than the final therapeutically effective dose.

Embodiment 54. The method of any of embodiments 1-53, wherein the ulotaront is titrated to a final therapeutically effective dose of 50, 75, or 100 mg/day over a period of from one week to six months, starting with a dose of 50% or less than the final dose.

Embodiment 55. The method of any of embodiments 1-54, wherein the ulotaront is administered orally.

Embodiment 56. The method of any one of embodiments 1-55, wherein the ulotaront is administered once daily in the fed or fasted state.

Embodiment 57. The method of any of embodiments 1-56, wherein the ulotaront is administered in multiple doses

Embodiment 58. The method of any of embodiments 1-57, wherein the ulotaront is administered as the free base or as a pharmaceutically acceptable salt.

Embodiment 59. The method of any of embodiments 1-58, wherein the ulotaront is administered as the hydrochloride salt.

Embodiment 60. A method of administering ulotaront to a human subject in need thereof with renal impairment, comprising a) administering ulotaront to the subject; b) measuring plasma concentration of ulotaront and of metabolite SEP-103; c) discontinuing ulotaront if the ratio of metabolite Cₘₐₓ to ulotaront Cₘₐₓ greater than 0.9 or if the ratio of metabolite AUC to ulotaront AUC greater than 1.8.

Embodiment 61. The method of embodiment 60, wherein the plasma concentration of ulotaront and of metabolite SEP-103 is measured for 96 hours.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated and are intended to be purely exemplary of the disclosure and are not intended to limit the scope of what the inventors regard as their invention.

### Example 1: A Phase 1, Open-Label, Parallel-Group Pharmacokinetic Study of SEP-363856 in Subjects with Severe Renal Impairment and End-Stage Renal Disease (ESRD) Compared to Subjects with Normal Renal Function

A pharmacokinetic study was undertaken in patients with severe renal impairment and ESRD to determine the effect of renal impairment on the pharmacokinetic profiles of SEP-363856 and SEP-383103. SEP-383103 was selected for evaluation because of earlier studies showing it to be a major metabolite of SEP-363856, and toxicological studies indicating no hERG inhibition from SEP-383103 concentrations up to 30 μM.

### Study Objectives:

### Primary Objective

To evaluate the pharmacokinetic (PK) profile of SEP-363856 in subjects with severe renal impairment and subjects with end-stage renal disease (ESRD) compared to subjects with normal renal function.

### Safety Objective

To evaluate the safety and tolerability of a single dose of SEP-363856 in subjects with severe renal impairment and subjects with ESRD compared to subjects with normal renal function.

### Secondary Objective

To evaluate the PK profile of metabolite SEP-383103 in subjects with severe renal impairment and subjects with ESRD compared to subjects with normal renal function.

### Study Endpoints:

The primary pharmacokinetic (PK) endpoints are the following PK parameters for plasma SEP-363856:
AUC_{0-∞} or AUC from time zero to a defined time (AUC₀₋ₜ), if appropriate
Cₘₐₓ

### Study Design:

This was a Phase 1, open-label, parallel-group study to determine the effect of renal impairment on the PK, safety and tolerability of a single oral dose of 25 mg of SEP-363856 compared to demographically-matched (age, sex, weight, race) healthy subjects with normal renal function. Renal function was assessed according to estimated glomerular filtration rate (eGFR) calculated using the Modification of Diet in Renal Disease (MDRD) equation at Screening and Check-in. Estimates calculated using the Cockcroft-Gault equation were also reported. Subjects in Group 1 had severe renal impairment not requiring dialysis and subjects in Group 2 had ESRD requiring dialysis.

Healthy matched controls with normal renal function were enrolled to match (age, sex, weight, race) across the 2 groups. A healthy control could be matched to more than 1 renally impaired subject. Potential subjects were screened to assess their eligibility to enter the study within 28 days (Day −28 to Day −3) prior to dose administration (Day 1). Each subject completed 1 or 2 treatment periods (1 treatment period for Group 1 and normal healthy subjects with normal renal function and 2 treatment periods for Group 2).

Subjects in Group 1 and the normal healthy subjects were admitted into the Clinical Research Unit (CRU) on Day −2 and confined to the CRU until discharge on Day 5. On the morning of Day 1, following an overnight fast of at least 8 hours, a single oral dose of 25 mg SEP-363856 was administered with approximately 240 mL of water. No food was allowed for 4 hours post-dose. Subjects in Group 1 returned to the CRU for a Follow-up visit approximately 7 days after discharge (i.e., Day 12±2).

Subjects in Group 2 followed their established dialysis schedule. They were admitted into the CRU on Day −2 and confined to the CRU until discharge on Day 5. In the morning of Day 1 of Period 1 (dialysis day), following an overnight fast of at least 8 hours, a single oral dose of 25 mg SEP-363856 was administered with approximately 240 mL of water at the start (approximately) of the dialysis session. No food was allowed for 4 hours post-dose.

On Day 1 of Period 2 (non-dialysis day), following a washout period of at least 7 days between dosing days (Day 1 of Period 1 and Day 1 of Period 2) and an overnight fast of at least 8 hours, a single oral dose of 25 mg SEP-363856 was administered. No food was allowed for 4 hours post-dose. Subjects in Group 2 returned to the CRU for a Follow-up visit approximately 7 (±2) days after dosing in Period 2.

### Number of Subjects:

8 subjects with severe renal impairment and 8 subjects with ESRD were enrolled. 12 healthy matched controls with normal renal function were enrolled. The sample size was not based on power consideration, but the number of subjects chosen for this study was based on the standard number of subjects used in similar studies.

### Main Criteria for Subject Inclusion:

Male and female subjects aged between 18 and 70 years (inclusive) with a body mass index (BMI) between 18.0 and 40.0 kg/m² (inclusive). Subjects were in good health except for specific inclusion criteria related to the status of renal impairment.

### Investigational Product, Dosage and Mode of Administration:

A SEP-363856 25 mg tablet was given orally administered with 240 mL of water in the morning on Day 1 (in each period in Group 2) after an overnight fast. Subjects fasted for a minimum of 8 hours before, and an additional 4 hours post-dose (glucose tablets could be administered, if needed, at the discretion of the Investigator); intake of water was not restricted except for the period from 1-hour pre-dose through 1-hour post-dose.

### Duration of Treatment:

### Group 1

1 day in Group 1; subjects received SEP-363856 25 mg on 1 dosing occasion (Day 1)

### Group 2

2 days in Group 2; subjects received SEP-363856 25 mg on 2 dosing occasions (Day 1 of each period)

### Results

Ulotaront exposure increase was relatively small and manageable. Preliminary analyses revealed that ulotaront produced 1.1×Cₘₐₓ and 1.6×AUC in subjects with end-stage renal impairment.

The final results showed that, in subjects with severe renal impairment, SEP-363856 mean exposure was generally comparable to healthy subjects; geometric mean AUC_{0-∞} increased 1.37-fold [geometric LS mean ratio (90% CI):136.99% (109.17%, 171.90%)] with no change in Cₘₐₓ [107.19% (85.34%, 134.63%)] and comparable Tₘₐₓ. However, metabolite SEP-383103 exposure surprisingly increased dramatically in patients with severe renal impairment. A strong correlation between SEP-383103 exposure (AUC_{0-∞} and AUC₀₋ₗₐₛₜ) and renal function was observed. Specifically, in subjects with severe renal impairment, metabolite SEP-383103 exposure (AUC₀₋ₗₐₛₜ and Cₘₐₓ) increased approximately 6.97- and 2.38-fold, respectively, compared to healthy subjects, and median Tₘₐₓ was delayed substantially compared to Tₘₐₓ in healthy subjects. Dialysis had a negligible impact on SEP-363856 exposure.

### Example 2: Determination of Renal Impairment Cut-Off at which Ulotaront should not be Administered Due to Potential for Exposure to Levels of SEP-103 Exceeding Those Studied and Deemed Safe in Preclinical Toxicology Studies

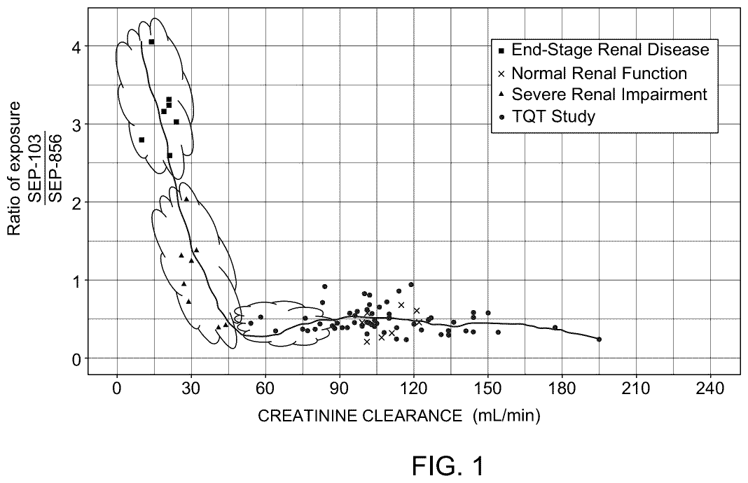

In order to determine the level of renal impairment at which ulotaront should not be administered, due to the potential for exposure to levels of SEP-383103 exceeding those studied and deemed safe in preclinical toxicology studies, a preliminary analysis of blood concentrations of SEP-856 and SEP-103 from three separate clinical studies was undertaken, and the ratio of SEP-103 exposure to SEP-856 exposure graphically plotted against creatinine clearance rates. The three studies from which blood concentrations were derived were:
Study 361-112 (the study reported in Example 1)
Study 361-113 (a study in hepatically impaired patients who were free of neuropsychiatric disorders, receiving 25 mg SEP-856)
Study 361-114 (a TQT study in schizophrenia patients receiving 150 mg SEP-856 to address potential for QTc prolongation).
The results of the evaluation are reported in FIG. 1.

### Example 3: A Phase 1, Open-Label, Parallel-Group Pharmacokinetic Study of SEP-856 in Subjects with Mild and Moderate Renal Impairment Compared to Subjects with Normal Renal Function

This was a Phase 1, open-label, parallel-group study to determine the effect of renal impairment on the pharmacokinetics, safety and tolerability of a single oral dose of SEP-856 25 mg compared to healthy subjects with normal renal function. Renal function was assessed according to estimated glomerular filtration rate (eGFR) calculated using the Modification of Diet in Renal Disease (MDRD) equation at Screening and Check-in.

Healthy controls with normal renal function were enrolled in an effort to approximately match demographics (age, sex, weight, race) across the 2 groups. Potential subjects were screened to assess their eligibility to enter the study within 28 days (Day −28 to Day −3) prior to dose administration (Day 1).

Nine healthy controls, 9 subjects with mild renal impairment, and 6 subjects with moderate renal impairment were enrolled.

Male and female subjects aged between 18 and 70 years (inclusive) with a body mass index (BMI) between 18.0 and 40.0 kg/m² (inclusive) were eligible for study inclusion. Subjects were in good health except for specific inclusion criteria related to the status of renal impairment.

A SEP-856 25 mg tablet was orally administered with 240 mL of water in the morning on Day 1 after an overnight fast. Subjects fasted for a minimum of 8 hours before dosing, and an additional 4 hours post-dose; intake of water was restricted except for the period from 1-hour pre-dose through 1-hour post-dose.

Blood and urine samples were collected for the analysis of concentrations of SEP-856 and its metabolite, SEP-103.

Primary endpoints included the following PK parameters for plasma SEP-363856 and its metabolite SEP-383103:
Area under the plasma concentration-time curve (AUC) from time zero to infinity (AUC_{0-∞}) or AUC from time zero to a defined time (AUC₀₋ₜ), if appropriate
Maximum observed plasma concentration (Cₘₐₓ).
Time to the maximum observed concentration (Tₘₐₓ).
Metabolite-to-parent ratio based on Cₘₐₓ and AUC (MR_{Cmax} and MR_{AUC}).

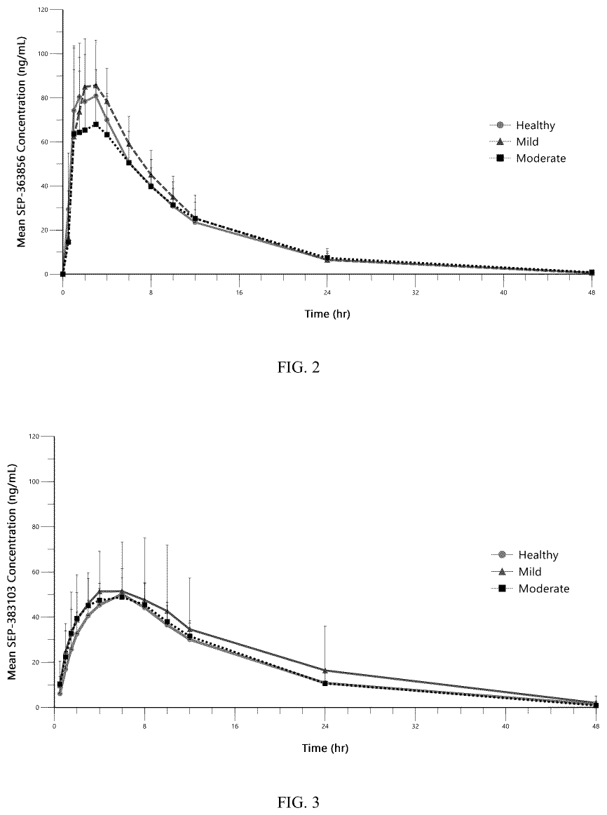

Preliminary results: NCA parameters AUC_{0-infinity}, Cₘₐₓ, and Tₘₐₓ, along with metabolite to parent ratios, are reported in Table 1 and in FIG. 2 and FIG. 3. Based on preliminary evaluation of the data, mean AUC for the parent drug SEP-856 appears unchanged due to mild (60-90 mL/min GFR) or moderate (30-60 mL/min GFR) impairment relative to healthy normal (>90 mL/min). Metabolite to parent ratios also appear unchanged across impairment groups. A similar observation can be made for Cₘₐₓ, which also appears unaffected by renal impairment, as is the metabolite to parent ratio. Median Tₘₐₓ for the metabolite is delayed by several hours in all test groups relative to parent Tₘₐₓ.

TABLE 1

| | | Renal | | SEP-856 | | SEP-103 | | Metabolite/Parent |
|---|---|---|---|---|---|---|---|---|
| | | Status | N | Mean | Std Dev | Mean | Std Dev | Ratio |
| AUC | hr*ng/mL | Healthy | 9 | 877 | 155 | 853 | 194 | 0.97 |
| AUC | hr*ng/mL | Mild | 9 | 927 | 246 | 1055 | 777 | 1.14 |
| AUC | hr*ng/mL | Moderate | 6 | 858 | 296 | 873 | 193 | 1.02 |

| | | | | SEP-856 | | SEP-103 | | |
|---|---|---|---|---|---|---|---|---|
| | | | N | Mean | Std Dev | Mean | Std Dev | |
| Cₘₐₓ | ng/mL | Healthy | 9 | 95.1 | 14.9 | 50.8 | 10.7 | 0.53 |
| Cₘₐₓ | ng/mL | Mild | 9 | 97.0 | 19.3 | 57.0 | 25.6 | 0.59 |
| Cₘₐₓ | ng/mL | Moderate | 6 | 75.9 | 21.0 | 51.7 | 11.4 | 0.68 |

| | | | | SEP-856 | SEP-103 |
|---|---|---|---|---|---|
| | | | N | Median | Median |
| Tₘₐₓ | hr | Healthy | 9 | 2 | 6 |
| Tₘₐₓ | hr | Mild | 9 | 2 | 6 |
| Tₘₐₓ | hr | Moderate | 6 | 2.5 | 5 |

In a study with 24 subjects, all 24 subjects dosed with SEP-363856 were included in the PK Population, including eight healthy control subjects with normal renal function, eight subjects with mild renal impairment ([eGFR: 60-89 mL/min/1.73 m²]), and eight subjects with moderate renal impairment ([eGFR: 30-59 mL/min/1.73 m²]) based on eGFR in mL/min/1.73 m² units. All 24 subjects completed the study.

Final Results: Results were obtained by fitting an ANOVA model to the natural log-transformed PK parameter, with renal function group as fixed effect and subject as random effect. Results were back transformed to obtain the geometric LS mean ratio and 90% CI.

SEP-363856 PK parameters following administration of a single dose of SEP-363856 25 mg are summarized by renal function group in Table 2. The evaluation of the effect of renal impairment on the primary SEP-363856 PK parameters AUC_{0-∞}, and Cₘₐₓ is summarized in Table 3.

TABLE 2
SEP-363856 Plasma Pharmacokinetic Parameters
by Renal Function Group (PK Population)

| | Renal Function Group | | | | | |
|---|---|---|---|---|---|---|
| Parameter | Healthy | | Mild | | Moderate | |
| (unit) | (N, n = 8) | | (N, n = 8) | | (N, n = 8) | |
| Cₘₐₓ (ng/mL) | 95.46 | (15.4) | 92.95 | (21.9) | 78.39 | (34.5) |
| Tₘₐₓ (h)^{a} | 1.775 | (0.95, 4.02) | 2.500 | (1.00, 4.00) | 2.000 | (1.00, 4.00) |
| AUC_{0-∞} | 830.4 | (17.1) | 793.9 | (15.4) | 813.5 | (43.3) |
| (ng*h/mL) | | | | | | |
| AUC₀₋ₗₐₛₜ | 829.8 | (17.1) | 793.4 | (15.5) | 812.8 | (43.3) |
| (ng*h/mL) | | | | | | |
| T_{1/2} (h) | 8.676 | (31.7) | 9.188 | (31.1) | 8.520 | (29.1) |
| CL/F (L/h) | 30.11 | (17.1) | 31 49 | (15.4) | 30.73 | (43.3) |
| V_{z}/F (L) | 376.8 | (30.0) | 417.4 | (22.5) | 377.8 | (27.1) |

Abbreviations:
AUC_{0-∞} = area under the plasma concentration-time curve from time zero to infinity; AUC₀₋ₗₐₛₜ = area under the plasma concentration-time curve from time zero to last quantifiable concentration; CL/F = apparent clearance; Cₘₐₓ = maximum observed plasma concentration; eGFR = estimated glomerular filtration rate; GeoCV % = geometric coefficient of variation; GeoMean = geometric mean; Max = maximum; Min = minimum; N = number of subjects in the group; n = number of subjects in the analysis; PK = pharmacokinetic; T_{1/2} = terminal elimination half-life; Tₘₐₓ = time of Cₘₐₓ; V_{z}/F = apparent volume of distribution.
Note:
Unless otherwise specified, results are presented as GeoMean (GeoCV %). Renal function group categorization was based on eGFR in mL/min/1.73 m2 units.
^{a}Results are presented as Median (Min, Max).

TABLE 3
Effect of Renal Impairment on SEP-363856 Exposure (PK Population)

| | Renal | | | | Geo LS | 90% CI of Geo |
|---|---|---|---|---|---|---|
| Parameter | Function | | Geo LS | | Mean Ratio | LS Mean Ratio |
| (unit) | Group | n | Mean | Comparison | (%) | (%) |
| AUC_{0-∞} | Healthy | 8 | 830.4 | | | |
| (ng*h/mL) | Mild | 8 | 793.9 | Mild vs Heathy | 95.61 | (75.57, 120.95) |
| | Moderate | 8 | 813.5 | Moderate vs Healthy | 97.96 | (77.43, 123.93) |
| Cₘₐₓ | Healthy | 8 | 95.46 | | | |
| (ng/mL) | Mild | 8 | 92.95 | Mild vs Healthy | 97.37 | (78.74, 120.41) |
| | Moderate | 8 | 78.39 | Moderate vs Healthy | 82.11 | (66.40, 101.55) |

Abbreviations: ANOVA = analysis of variance; AUC_{0-∞} = area under the plasma concentration-time curve from time zero to infinity; CI = confidence interval; Cₘₐₓ = maximum observed plasma concentration; eGFR = estimated glomerular filtration rate; Geo = geometric; LS = least squares; n = number of subjects in the analysis; PK = pharmacokinetic.
Note:
Renal function group categorization was based on eGFR in mL/min/1.73 m² units. Results were obtained by fitting an ANOVA model to the natural log-transformed PK parameter, with renal function group as fixed effect and subject as random effect. Results were back transformed to obtain the geometric LS mean ratio and 90% CI.

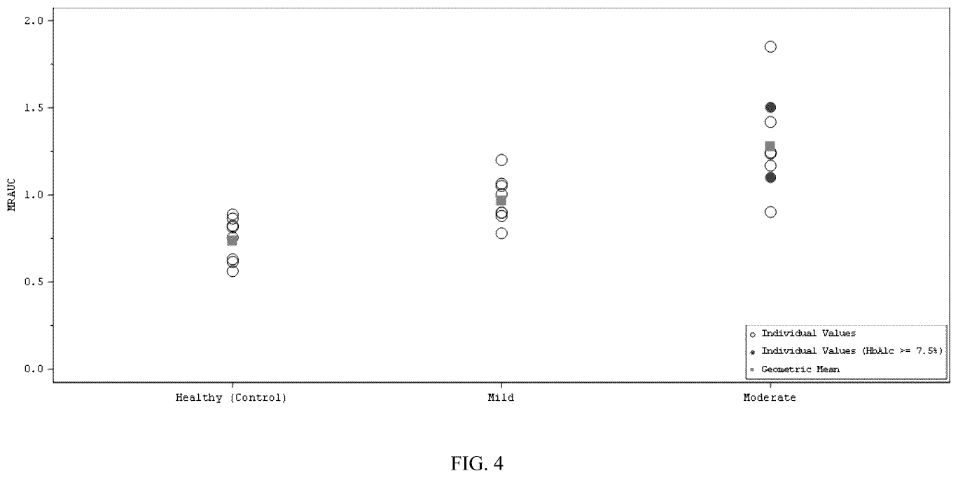

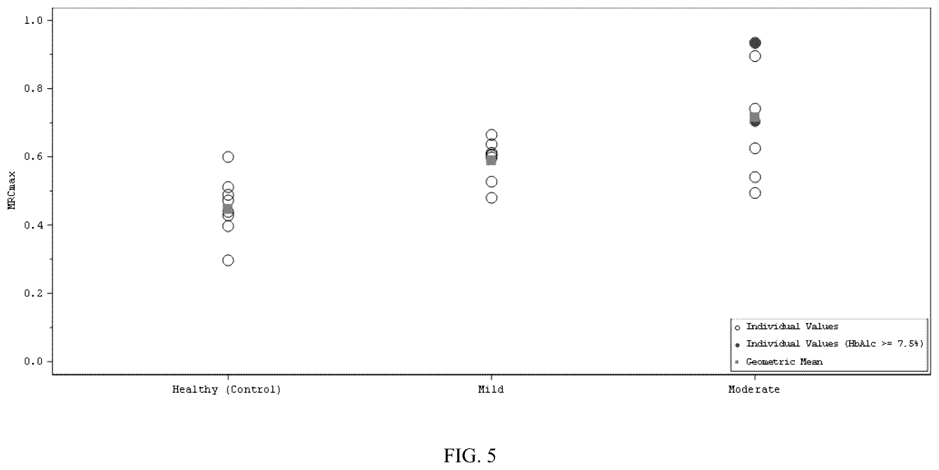

SEP-383103 PK parameters following administration of a single dose of SEP-363856 25 mg were summarized by renal function group in Table 4. The evaluation of the effect of renal impairment on the primary SEP-383103 PK parameters AUC_{0-∞}, and Cₘₐₓ is summarized in Table 5. Individual and geometric mean MR_{AUC} and MR_{Cmax} are presented by renal function group in FIG. 4 and FIG. 5, respectively. There is a significant correlation between exposure to SEP-383103 (AUC_{0-∞}, and Cₘₐₓ) and the degree of renal impairment. Renal function group categorization was based on eGFR in mL/min/1.73 m² units. (eGFR=estimated glomerular filtration rate; AUC_{0-∞} area under the plasma concentration-time curve from time zero to infinity; MR_{AUC}=metabolite-to-parent ratio based on AUC_{0-∞}; PK=pharmacokinetic; Cₘₐₓ=maximum observed plasma concentration; MR_{Cmax}=metabolite-to-parent ratio based on Cₘₐₓ.)

TABLE 4
SEP-383103 Plasma Pharmacokinetic Parameters
by Renal Function Group (PK Population)

| | Renal Function Group | | | | | |
|---|---|---|---|---|---|---|
| Parameter | Healthy | | Mild | | Moderate | |
| (unit) | (N, n = 8) | | (N, n = 8) | | (N, n = 8) | |
| Cₘₐₓ (ng/mL) | 42.81 | (11.8) | 54.98 | (19.3) | 56.30 | (46.9) |
| Tₘₐₓ (h)^{a} | 3 975 | (2.95, 6 00) | 5.000 | (2.00, 6.00) | 6.000 | (3 00, 9.98) |
| AUC_{0-∞} | 613.3 | (20.3) | 769.5 | (17.0) | 1043 | (57.9) |
| (ng*h/mL) | | | | | | |
| AUC₀₋ₗₐₛₜ | 608.1 | (20.2) | 763.7 | (17.0) | 1038 | (58.0) |
| (ng*h/mL) | | | | | | |
| T_{1/2} (h) | 7.057 | (14.1) | 6.231 | (8.8) | 7.633 | (21.5) |
| MR_{Cmax} | 0 4462 | (20.9) | 0.5885 | (10.6) | 0.7146 | (25.1) |
| MR_{AUC} | 0.7348 | (17.6) | 0.9643 | (13.7) | 1.276 | (21.9) |

Abbreviations:
AUC_{0-∞} = area under the plasma concentration-time curve from time zero to infinity; AUC₀₋ₗₐₛₜ = area under the plasma concentration-time curve from time zero to last quantifiable concentration; Cₘₐₓ = maximum observed plasma concentration; eGFR = estimated glomerular filtration rate; GeoCV % = geometric coefficient of variation; GeoMean = geometric mean; Max = maximum; Min = minimum; MR_{AUC} = metabolite-to-parent ratio based on AUC_{0-∞}; MR_{Cmax} = metabolite-to-parent ratio based on Cₘₐₓ; N = number of subjects in the group; n = number of subjects in the analysis; PK = pharmacokinetic; T_{1/2} = terminal elimination half-life; Tₘₐₓ = time of Cₘₐₓ.
Note:
Results are presented as GeoMean (GeoCV %). Renal function group categorization was based on eGFR in mL/min/1.73 m² units.
^{a}Results are presented as Median (Min, Max).

TABLE 5
Effect of Renal Impairment on SEP-383103 Exposure (PK Population)

| | Renal | | | | Geo LS | 90% CI of Geo |
|---|---|---|---|---|---|---|
| Parameter | Function | | Geo LS | | Mean Ratio | LS Mean Ratio |
| (unit) | Group | n | Mean | Comparison | (%) | (%) |
| AUC_{0-∞} | Healthy | 8 | 613.3 | | | |
| (ng*h/mL) | Mild | 8 | 769.5 | Mild vs Healthy | 125.47 | (93.22, 168.87) |
| | Moderate | 8 | 1043 | Moderate vs Healthy | 170.12 | (126.40, 228.97) |
| Cₘₐₓ | Healthy | 8 | 42.81 | | | |
| (ng/mL) | Mid | 8 | 54.98 | Mild vs Healthy | 128.43 | (100.23, 164.55) |
| | Moderate | 8 | 56.30 | Moderate vs Healthy | 131.50 | (102.64, 168.49) |

Abbreviations: ANOVA = analysis of variance; AUC_{0-∞} = area under the plasma
concentration-time curve from time zero to infinity; CI = confidence interval; Cₘₐₓ = maximum
observed plasma concentration; eGFR = estimated glomerular filtration rate; Geo = geometric;
LS = least squares; n = number of subjects in the analysis; PK = pharmacokinetic.
Note:
Renal function group categorization was based on eGFR in mL/min/1.73 m² units. Results were obtained by fitting an ANOVA model to the natural log-transformed PK parameter, with renal function group as fixed effect and subject as random effect. Results were back transformed to obtain the geometric LS mean ratio and 90% CI.

Taken together, the results showed increasing plasma exposure to metabolite SEP-383103, and to a smaller extent SEP-363856, as renal function progressively decreases from healthy normal subjects to ESRD patients. In subjects with mild renal impairment, SEP-363856 exposure (AUC_{0-∞} and Cₘₐₓ) was similar to healthy subjects. Metabolite SEP-383103 AUC_{0-∞} and Cₘₐₓ increased to a small extent (1.25- and 1.28-fold, respectively), with no change in Tₘₐₓ. In subjects with moderate renal impairment, SEP-363856 AUC_{0-∞} was similar to healthy subjects and there was no increase in Cₘₐₓ. Metabolite SEP-383103 AUC_{0-∞} and Cₘₐₓ increased 1.70- and 1.32-fold, respectively, and median Tₘₐₓ was delayed by 2 hours compared to healthy subjects. No reduction in urine recovery was observed in subjects with mild or moderate renal impairment. Overall, exposure to the pharmacologically inactive metabolite SEP-383103 following a single dose of SEP-363856 25 mg in subjects with eGFR as low as 30 mL/min/1.73 m² was well within the levels studied in nonclinical toxicology studies.

Safety: A single dose of SEP-363856 25 mg was well tolerated by healthy subjects, subjects with mild renal impairment, and subjects with moderate renal impairment.

### REFERENCES CITED

1. Cohen L. et al., Update on Psychotropic Medication Use in Renal Disease. Psychosomatics 2004; 45:34-48.
2. Dedic N. et al., SEP-363856, a Novel Psychotropic Agent with a Unique, Non-D2 Receptor Mechanism of Action. J Pharmacol Exp Ther 371:1-14, October 2019.
3. Galluppi G. et al., Population pharmacokinetic analysis of ulotaront in subjects with schizophrenia. CPT Pharmacometrics Syst Pharmacol. 2021; 10:1245-1254.
4. Gardner D. et al., International Consensus Study of Antipsychotic Dosing. 2014 American Journal of Psychiatry 2010; 167 (6): 686-693.
5. Koblan K. et al., A Non-D2-Receptor-Binding Drug for the Treatment of Schizophrenia. N Engl J Med 2020; 382:1497-506.
6. Levy G., Pharmacokinetics in renal disease. Am J Med. 1977; 62 (4): 461-465.
7. Xiao G. et al., In Vitro ADME and Preclinical Pharmacokinetics of Ulotaront, a TAAR1/5-HT_{1A} Receptor Agonist for the Treatment of Schizophrenia. Pharmaceutical Research (2022) 39:837-850.

Throughout this application, various publications are referenced. The disclosures of these publications in their entireties are hereby incorporated by reference into this application in order to more fully describe the state of the art to which this disclosure pertains. It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of administering ulotaront to a human subject in need thereof with renal impairment or declining renal function, comprising:
a) measuring or evaluating the subject's renal function; and
b) administering ulotaront to the subject if the subject has a creatinine clearance rate greater than or equal to 30 mL/min or an eGFR greater than or equal to 30 mL/min/1.73 m².

2. (canceled)

3. A method of administering ulotaront to a human subject in need thereof, wherein the subject is on ulotaront with declining renal function, comprising:
a) measuring or evaluating the subject's renal function;
b) administering ulotaront while the subject has a creatinine clearance rate greater than or equal to 30 mL/min or an eGFR greater than or equal to 30 mL/min/1.73 m²; and
c) upon the subject having a creatinine clearance rate less than 30 mL/min or an eGFR less than 30 mL/min/1.73 m², or kidney failure, not administering the ulotaront.

4. (canceled)

5. (canceled)

6. A method of reducing exposure to SEP-103 during chronic administration of ulotaront, in a human subject in need thereof with renal impairment or with declining renal function, comprising:
a) measuring or evaluating the subject's renal function;
b) administering ulotaront to the subject if the subject has a renal function above a threshold; and
c) not administering ulotaront to the subject if the subject has a renal function below the threshold.

7. (canceled)

8. A method of administering ulotaront to a human subject in need thereof, wherein the subject is on ulotaront administration with declining renal function, comprising:
a) measuring or evaluating the subject's renal function;
b) administering ulotaront to the subject while the subject has a renal function above a threshold; and
c) not administering ulotaront if the subject has a renal function below the threshold.

9. The method of claim 1, comprising administering ulotaront to the subject if the subject has a creatinine clearance rate greater than or equal to 45 mL/min or an eGFR greater than or equal to 45 mL/min/1.73 m², or administering ulotaront to the subject if the subject has a creatinine clearance rate greater than or equal to 60 mL/min or an eGFR greater than or equal to 60 mL/min/1.73 m².

10. (canceled)

11. The method of claim 8,
wherein the subject is non-renally impaired.

12. The method of claim 6, wherein the subject is renally impaired.

13. The method of claim 6, wherein the subject is mildly, moderately, or severely renally impaired, or with kidney failure.

14. The method of claim 8, wherein the subject is renally impaired.

15. The method of claim 8, wherein the subject is mildly, moderately, or severely renally impaired, or with kidney failure.

16. (canceled)

17. (canceled)

18. The method of claim 1, wherein the creatinine clearance rate or threshold prevents blood concentrations of SEP-103 from exceeding levels studied and deemed to be safe in preclinical toxicology studies, or wherein the creatinine clearance rate or threshold prevents exposure to SEP-103 from exceeding levels greater than those studied and deemed to be safe in preclinical toxicology studies.

19. (canceled)

20. The method of claim 6, wherein the threshold is the upper bound of moderate renal impairment, or is the upper bound of severe renal impairment, or is the upper bound of kidney failure.

21. The method of claim 8, wherein the threshold is the upper bound of moderate renal impairment, or is the upper bound of severe renal impairment, or is the upper bound of kidney failure.

22. (canceled)

23. The method of claim 6, wherein the threshold is a creatinine clearance rate of 60 mL/min, or is a creatinine clearance rate of 45 mL/min, or is a creatinine clearance rate of 30 mL/min, or is a creatinine clearance rate of 15 mL/min.

24. The method of claim 8, wherein the threshold is a creatinine clearance rate of 60 mL/min, or is a creatinine clearance rate of 45 mL/min, or is a creatinine clearance rate of 30 mL/min, or is a creatinine clearance rate of 15 mL/min.

25. (canceled)

26. (canceled)

27. The method of claim 6, wherein the threshold is an eGFR of 60 mL/min/1.73 m², or is an eGFR of 45 mL/min/1.73 m², or is an eGFR of 30 mL/min/1.73 m², or is an eGFR of 15 mL/min/1.73 m².

28. The method of claim 8, wherein the threshold is an eGFR of 60 mL/min/1.73 m², or is an eGFR of 45 mL/min/1.73 m², or is an eGFR of 30 mL/min/1.73 m², or is an eGFR of 15 mL/min/1.73 m².

29. (canceled)

30. (canceled)

31. (canceled)

32. The method of claim 1, further comprising providing an alternative neuropsychiatric medication suitable for administration to human subjects with creatinine clearance below 30 mL/min or eGFR below 30 mL/min/1.73 m²; or further comprising, if ulotaront is not administered, administering an alternative neuropsychiatric medication suitable for administration to human subjects with creatinine clearance below 30 mL/min or eGFR below 30 mL/min/1.73 m².

33. (canceled)

34. (canceled)

35. The method of claim 32, wherein the neuropsychiatric medication and its major metabolites are excreted or metabolized predominantly outside the kidneys.

36. The method of claim 6, wherein the ulotaront is accompanied by written instructions instructing non-administration of the ulotaront if the subject has renal function below the threshold, further comprising, in response to the measurement or evaluation of renal function, based on the written instructions:
i) administering ulotaront to the subject if the subject has renal function above the threshold; and
ii) not administering ulotaront to the subject if the subject has renal function below the threshold.

37. The method of claim 8, wherein the ulotaront is accompanied by written instructions instructing non-administration of the ulotaront if the subject has renal function below the threshold, further comprising, in response to the measurement or evaluation of renal function, based on the written instructions:
i) administering ulotaront to the subject if the subject has renal function above the threshold; and
ii) not administering ulotaront to the subject if the subject has renal function below the threshold.

38. (canceled)

39. (canceled)

40. The method of claim 1, wherein the subject is on a preexisting regimen of a neuropsychiatric therapy and, when the ulotaront is administered, it is administered in addition to the preexisting regimen; or wherein the subject is on a preexisting regimen of a neuropsychiatric therapy and, when the ulotaront is administered, it is administered instead of the preexisting regimen.

41. (canceled)

42. The method of claim 1, wherein the subject is on a preexisting regimen of a neuropsychiatric therapy, and the preexisting regimen produces an inadequate therapeutic response or a clinically significant adverse event.

43. The method of claim 1, wherein the subject is on a preexisting regimen of a neuropsychiatric therapy, wherein the preexisting regimen produces a clinically significant adverse event selected from akathisia, blood prolactin abnormal, blood prolactin increased, blood triglycerides increased, body mass index increased, bradykinesia, bruxism, cogwheel rigidity, dermatillomania, diabetes mellitus, drooling, dyskinesia, dyslipidaemia, dyssomnia, dystonia, electrocardiogram QT prolonged, enuresis, excessive eye blinking, extrapyramidal disorder, galactorrhoea, glucose tolerance impaired, glycosuria, hyperkinesia, hyperprolactinaemia, impaired fasting glucose, increased appetite, metabolic syndrome, muscle rigidity, nuchal rigidity, obesity, obsessive-compulsive disorder, oculogyric crisis, oromandibular dystonia, orthostatic hypertension, overweight, pancreatitis chronic, parkinsonian gait, parkinsonism, psychomotor retardation, restless legs syndrome, restlessness, salivary hypersecretion, sedation, sexual dysfunction, tardive dyskinesia, tic, tongue biting, tongue spasm, torticollis, type 2 diabetes mellitus, and weight increase.

44. (canceled)

45. The method of claim 1, wherein the subject has a condition selected from schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, aggression, delirium, Parkinson's psychosis, excitative psychosis, Tourette's syndrome, organic or NOS psychosis, seizure, agitation, post-traumatic stress disorder, behavior disorder, neurodegenerative disease, Alzheimer's disease, Parkinson's disease, dyskinesias, Huntington's disease, dementia, mood disorder, anxiety, affective disorders (e.g., depression, e.g., major depressive disorder and dysthymia; bipolar disorder, e.g., bipolar depressive disorder; manic disorder; seasonal affective disorder; and attention deficit disorder (ADD) and attention deficit hyperactivity disorder (ADHD)), obsessive-compulsive disorder, vertigo, epilepsy, pain (e.g., neuropathic pain, sensitization accompanying neuropathic pain, and inflammatory pain), fibromyalgia, migraine, cognitive impairment, movement disorder, restless leg syndrome (RLS), multiple sclerosis, sleep disorder, sleep apnea, narcolepsy, excessive daytime sleepiness, jet lag, drowsy side effect of medications, insomnia, substance abuse or dependency (e.g., nicotine, cocaine), addiction, eating disorder, sexual dysfunction, hypertension, emesis, Lesche-Nyhane disease, Wilson's disease, autism, Huntington's chorea, and premenstrual dysphoria; or wherein the subject has a condition selected from psychosis, mood disorders, anxiety disorders, neurocognitive disorders, substance use disorders, and sleep disorders; or wherein the subject has a condition selected from schizophrenia, depression (including aMDD), and anxiety (including GAD).

46. (canceled)

47. (canceled)

48. The method of claim 1, wherein the ulotaront is administered at a therapeutically effective dose.

49. The method of claim 1, further comprising administering or providing the ulotaront as a therapeutically effective dose, which is the same regardless of whether the human subject is non-renally impaired, mildly renally impaired, or moderately renally impaired, and administering ulotaront at the therapeutically effective dose regardless of whether the human subject is non-renally impaired, mildly renally impaired, or moderately renally impaired.

50. The method of claim 1, wherein the ulotaront is administered in a dose of from 10 to 150 or 25 to 100 mg/day of ulotaront or a pharmaceutically acceptable salt thereof; and/or wherein the ulotaront is administered in a dose selected from 25, 50, 75, 100, 125, and 150 mg/day of ulotaront or a pharmaceutically acceptable salt thereof.

51. (canceled)

52. The method of claim 1, wherein the subject is on a preexisting regimen of neuropsychiatric therapy, further comprising administering the preexisting regimen and the ulotaront during a tapering period.

53. The method of claim 1, wherein the ulotaront is titrated to a final therapeutically effective dose over a period of from one week to six months, starting with a dose of 50% or less than the final therapeutically effective dose; and/or wherein the ulotaront is titrated to a final therapeutically effective dose of 50, 75, or 100 mg/day over a period of from one week to six months, starting with a dose of 50% or less than the final dose.

54. (canceled)

55. The method of claim 1, wherein the ulotaront is administered orally.

56. The method of claim 1, wherein the ulotaront is administered once daily in the fed or fasted state.

57. The method of claim 1, wherein the ulotaront is administered in multiple doses.

58. The method of claim 1, wherein the ulotaront is administered as the free base or as a pharmaceutically acceptable salt.

59. The method of claim 1, wherein the ulotaront is administered as the hydrochloride salt.

60. A method of administering ulotaront to a human subject in need thereof with renal impairment, comprising:
a) administering ulotaront to the subject;
b) measuring plasma concentration of ulotaront and of metabolite SEP-103; and
c) discontinuing ulotaront if the ratio of metabolite Cₘₐₓ to ulotaront Cₘₐₓ greater than 0.9 or if the ratio of metabolite AUC to ulotaront AUC greater than 1.8.

61. The method of claim 60, wherein said plasma concentration of ulotaront and of metabolite SEP-103 is measured for 96 hours.
